(19) United States
(12) Patent Application Publication
(10) Pub. No.: US 2022/0257517 A1
(43) Pub. Date: Aug. 18, 2022

(54) POWDER COMPOSITION COMPRISING A COPOLYMER MIXTURE AND A WATER-SOLUBLE CELLULOSE

(71) Applicant: Evonik Operations GmbH, Essen (DE)

(72) Inventors: Ashish Guha, Mumbai (IN); Sonam Singh, Thane (IN); Suresh Doke, Navi Mumbai, Maharashtra (IN); Shraddha Joshi, Thane (IN); Manfred Aßmus, Bickenbach (DE); Jitendra Amrutkar, Thane ( West) (IN)

(73) Assignee: Evonik Operations GmbH, Essen (DE)

(21) Appl. No.: 17/596,434

(22) PCT Filed: Jun. 8, 2020
(86) PCT No.: PCT/EP2020/065806

§ 371 Date: Dec. 10, 2021

(30) Foreign Application Priority Data
Jun. 13, 2019 (IN) 201941023489

(51) Int. Cl. A61K 9/16; C08F 220/06; C08F 220/18; C08F 220/14; C08L 1/04; A23L 33/24; A23L 33/25; A61K 9/20

(52) U.S. Cl. CPC A61K 9/1635; A61K 9/1652; C08F 220/06; C08F 220/1802; C08F 220/14; C08L 1/04; C08L 2205/03; A23L 33/25; A61K 9/2095; C08L 2201/50; C08L 2207/53; C08L 2205/02; A23L 33/24

(57) ABSTRACT

A powder composition contains 50 to 95% by weight of a copolymer mixture A of a copolymer 1 and a copolymer 2, and 50 to 5% by weight of a water-soluble cellulose B. The copolymer 1 contains 5 to 60% by weight of polymerized units of methacrylic acid and 95 to 40% by weight of C₁- to C₄-alkylesters of (meth)acrylic acid. The copolymer 2 contains more than 95 and up to 100% by weight of polymerized units of C₁- to C₄-alkylesters of (meth)acrylic acid. The powder composition originates from the co-processing of the copolymer mixture A and the water-soluble cellulose B by a drying process of an aqueous dispersion, such as spray drying or freeze drying. Further processing leads to a compressed dosage form.

### FIELD OF THE INVENTION

The invention is in the field of pharmaceuticals and nutraceuticals, especially in the field of compressed dosage forms.

### BACKGROUND

Davood Hazanzadeh et al., “Thermal Treating of Acrylic Matrices as a Tool for Controlling Drug Release”, Chem. Pharm. Bull. 57(12) 1356-1362 (2009) highlights the requirement of thermal treatment for acrylic matrices, where thermal treatment leads to a more modified release profile compared to that of untreated one. Polymer chain movement and redistribution of the polymer in the tablet matrix structure after thermal treatment is the possible mechanism of drug release prolongation. The melting and resolidification of the polymer, due to the thermal treatment has apparently resulted in a redistribution of the polymer throughout the matrix and also in a change in the porosity of the tablet.

WO2012/171575A1 describes a coating composition suitable for pharmaceutical applications. The coating composition comprises core-shell polymers derived from a two-stage emulsion polymerization processes. EUDRAGIT® FL 30 D-55 (Evonik Nutrition & Care GmbH, Darmstadt, Germany), is a commercially available 30% by weight aqueous dispersion of a copolymer from a two-stage emulsion polymerization process, with a core of about 75% by weight, comprising polymerized units of about 70% by weight of ethyl acrylate and 30% by weight of methyl methacrylate, and a shell of about 25% by weight, comprising polymerized units of 50% by weight ethyl acrylate and 50% by weight methacrylic acid.

### SUMMARY OF THE INVENTION

Direct Compression is a simple form of oral dosage production as it contains only few process stages, leading to a shorter process cycle and faster production times. Directly compressible materials for modified release, which are available commercially, are either from natural or synthetic sources or from combinations thereof. Both these directly compressible excipient classes have drawbacks. Cellulose for example as obtained from natural source has an issue of batch to batch quality variation. On the other hand, the major drawback with acrylates (one of the most widely used polymer classes) is requirement of curing, usually 24 to 48 hours at elevated temperature, to get the desired stable release profile. Problems with the storage stability of acrylate-based sustained release (SR) matrices are also reported. Thus, there is a need to formulate a directly compressible SR system which overcomes the drawbacks as discussed.

The invention is concerned with a powder composition, comprising 50 to 95% by weight of a copolymer mixture A of a copolymer 1, comprising 5 to 60% by weight of polymerized units of methacylic acid and 95 to 40% by weight of C₁- to C₄-alkylesters of (meth)acrylic acid, and a copolymer 2, comprising more than 95 and up to 100% by weight of polymerized units of C₁- to C₄-alkylesters of (meth)acrylic acid, and 50 to 5% by weight of a water-soluble cellulose B. The powder composition may be processed to compressed dosage form with a stable active ingredient release profile without the need of a curing step. The powder composition originates from the co-processing of the copolymer mixture A and the water-soluble cellulose B by a drying process of an aqueous dispersion, such as spray drying or freeze drying. Further processing leads to a compressed dosage form.

### DETAILS OF THE INVENTION

### Powder Composition

The invention is concerned with a powder composition, comprising 50 to 95% by weight of a copolymer mixture A of a copolymer 1, comprising 5 to 60% by weight of polymerized units of methacylic acid and 95 to 40% by weight of C₁ to C₄-alkylesters of (meth)acrylic acid, and a copolymer 2, comprising more than 95 and up to 100% by weight of polymerized units of C₁- to C₄-alkylesters of (meth)acrylic acid, and 50 to 5% by weight of a water-soluble cellulose B. The powder composition may comprise 50 to 100 preferably 80 to 100% by weight of the copolymer mixture A and the water-soluble cellulose B. Optionally, pharmaceutical or nutraceutical excipients may be present in an amount of 0 to 50, preferably 0 to 20% by weight.

The average particle size d50 of the powder composition may be in the range of 1 to 2,000, preferably in the range of 1 to 1,000, most preferably in the range of 10 to 600 μm. The average diameter may be determined by sieving or by laser diffraction according to the United States Pharmacopeia 36 (USP) chapter <429> and European Pharmacopeia 7.0 (EP) chapter 2.9.31.

The laser diffraction method is based on the phenomenon that particles scatter light in all directions with an intensity pattern that is dependent on particle size. A representative sample, dispersed at an adequate concentration in a suitable liquid or gas, is passed through the beam of a monochromic light source, usually from a laser. The light scattered by the particles at various angles is measured by a multi-element detector, and numerical values relating to the scattering pattern are then recorded for subsequent analysis. The numerical scattering values are then transformed, using an appropriate optical model and mathematical procedure, to yield the proportion of total volume to a discrete number of size classes forming a volumetric particle size distribution (d50 describes a particle diameter corresponding to 50% of cumulative undersize distribution).

### Copolymer Mixture A

The copolymer mixture A is a mixture of a copolymer 1 and a copolymer 2.

The copolymer mixture A may comprise copolymer 1 and copolymer 2 as a mixture of separate copolymers 1 and 2 or as a mixture in the form of a core-shell copolymer.

### Separate Copolymers 1 and 2

The copolymer mixture A may comprise copolymer 1 and copolymer 2 as a mixture of separate copolymers 1 and 2.

### Copolymer 1

Copolymer 1 comprises 5 to 60% by weight polymerized units of methacylic acid and 95 to 40% by weight of C₁ to C₄-alkylesters of (meth)acrylic acid.

Suitable (meth)acrylate copolymers 1 may be polymerized from 40 to 60% by weight of methacrylic acid and 60 to 40% by weight of methyl methacrylate or 60 to 40% by weight of ethyl acrylate. EUDRAGIT® L 100 is a copolymer polymerized from 50% by weight of methyl methacrylate and 50% by weight of methacrylic acid. EUDRAGIT® L 100-55 is a copolymer polymerized from 50% by weight of ethyl acrylate and 50% by weight of methacrylic acid. EUDRAGIT® L 30 D-55 is an aqueous dispersion comprising 30% by weight EUDRAGIT® L 100-55.

Suitable (meth)acrylate copolymers 1 may be polymerized from 20 to 40% by weight of methacrylic acid and 80 to 60% by weight of methyl methacrylate. EUDRAGIT® S 100 is a copolymer polymerized from 70% by weight of methyl methacrylate and 30% by weight of methacrylic acid.

Suitable (meth)acrylate copolymers 1 may be polymerized from 10 to 30% by weight of methyl methacrylate, 50 to 70% by weight of methyl acrylate and 5 to 15% by weight of methacrylic acid. EUDRAGIT® FS is a copolymer polymerized from 25% by weight of methyl methacrylate, 65% by weight of methyl acrylate and 10% by weight of methacrylic acid. EUDRAGIT® FS 30 D is an aqueous dispersion comprising 30% by weight EUDRAGIT® FS.

### Copolymer 2

Copolymer 2 comprises more than 95 and up to 100% by weight of polymerized units of C₁- to C₄-alkylesters of (meth)acrylic acid.

The copolymer 2 may be a (meth)acrylate copolymer comprising polymerized units of 60 to 80% of ethyl acrylate and 40 to 20% by weight of methyl methacrylate. EUDRAGIT® NE and EUDRAGIT® NM are copolymers comprising polymerized units of 28 to 32% by weight of methyl methacrylate and 68 to 72% by weight of ethyl acrylate. Preference is given to (meth)acrylate copolymers which, according to WO 01/68767, have been prepared as dispersions using 1 to 10% by weight of a non-ionic emulsifier having an HLB value of 15.2 to 17.3. The latter offer the advantage that there is no phase separation with formation of crystal structures by the emulsifier (EUDRAGIT® NM type).

### Core-Shell Copolymer

The copolymer mixture A may comprise copolymer 1 and copolymer 2 as a mixture in the form of a core-shell copolymer. Suitable core-shell copolymers are known for instance from WO2012/171575A1.

The copolymer mixture A may comprise a mixture of (meth)acrylate copolymers in the form of a core-shell polymer from two (meth)acrylate copolymer(s) corresponding to copolymer 1 and copolymer 2 respectively. The copolymer mixture A may be a core-shell polymer, comprising 50 to 90, preferably 70 to 80% by weight of a core, comprising polymerized units of 60 to 80, preferably 65 to 75% by weight of ethyl acrylate and 40 to 20, preferably 35 to 25% by weight of methyl methacrylate, and 50 to 10, preferably 30 to 20% by weight of a shell, comprising polymerized units of 40 to 60, preferably 45 to 55% by weight of ethyl acrylate and 60 to 40, preferably 55 to 45% by weight of methacrylic acid. Thus, in this case, the core corresponds to a copolymer 2 and the shell to a copolymer 1.

A suitable core-shell polymer is EUDRAGIT® FL 30 D-55 (Evonik Nutrition & Care GmbH, Darmstadt, Germany), which is a commercially available 30% by weight aqueous dispersion of a copolymer from a two-stage emulsion polymerization process, with a core of about 75% by weight, comprising polymerized units of about 70% by weight of ethyl acrylate and 30% by weight of methyl methacrylate, and a shell of about 25% by weight, comprising polymerized units of 50% by weight ethyl acrylate and 50% by weight methacrylic acid.

### Water-Soluble Cellulose B

A water-soluble cellulose is a cellulose which is soluble in water at a concentration of 1% by weight at a temperature of 25° C.

The water-soluble cellulose B is preferably methyl cellulose, hydroxyethyl cellulose, hydroxyethyl methyl cellulose, hydroxypropyl cellulose and/or, most preferred hydroxypropyl methyl cellulose. The viscosity of the water-soluble cellulose may be in the range from about 1 to 5,000 mPa·s, when measured as 1% aqueous solution or colloidal dispersion (weight/weight) at 25° C.

### Process for Preparing a Compressed Dosage Form

Disclosed is a process for preparing a compressed dosage form, preferably a tablet, comprising the pharmaceutical or nutraceutical composition comprising the steps i) to iv): i) providing an aqueous dispersion of the copolymer mixture A and the water-soluble cellulose B, ii) drying, preferably spray drying or freeze drying, of the aqueous dispersion to gain a powder, iii) mixing the powder with one or more biologically active ingredient(s) and one or more pharmaceutical or nutraceutical excipient(s) to obtain a mixture for compression, iv) compressing the mixture for compression into a form to obtain the compressed dosage form.

### Step i)

In step i) an aqueous dispersion of the copolymer mixture A and the water-soluble cellulose B is provided.

### Step ii)

In step ii) the aqueous dispersion from step i) is dried to gain a powder composition as claimed.

Preferred is spray drying or freeze drying.

Spray drying may be performed at an inlet temperature of 30 to 60, preferably 35 to 55° C.

Freeze drying is performed with a drying circle step for 4 to 16 hours at 350 to 450 mTorr, starting from −40 to −25° C. and increasing stepwise or continuously to a final temperature of 15 to 30° C.

The average particle size d50 of the powder may be in the range of 1 to 2,000, preferably in the range of 1 to 1,000, most preferably in the range of 10 to 600 μm. The average diameter may be determined by sieving or by laser diffraction according to the United States Pharmacopeia 36 (USP) chapter <429> and European Pharmacopeia 7.0 (EP) chapter 2.9.31.

Step iii)

In step iii) the powder from step ii) is mixed with one or more biologically active ingredient(s) and one or more pharmaceutical or nutraceutical excipient(s) to obtain a mixture for compression.

### Step iv)

In step iv) the mixture for compression from step iii) is compressed in a form to obtain a compressed dosage form, preferably a compressed tablet.

The weight of the compressed tablet may be from 2 to 2,000 mg, preferably 30 to 1,200 mg and most preferably from 100 to 800 mg.

The forces applied in the compression process may be in the range of 1 to 20 kN, preferably 2 to 10 kN. The resulting tablet hardness may be in the range of 10 to 250, preferably 50 to 150 N. Methods and equipment for determining the hardness of a tablet are well known to a skilled person in the field of pharmacy, galenic or nutraceutical technology.

### Compressed Dosage Form

The compressed dosage form as disclosed, preferably a tablet, is comprising one or more pharmaceutically or nutraceutically active ingredient(s) and a powder composition and one or more pharmaceutical or nutraceutical excipient(s).

Preferably, the compressed dosage form may comprise 1 to 50% by weight of the one or more biologically active ingredient(s), 10 to 70% by weight of the powder composition and 10 to 89% by weight of the one or more pharmaceutical or nutraceutical excipient(s). The one or more biologically active ingredient(s), the powder composition and the one or more pharmaceutical or nutraceutical excipient(s) may add up to 100%.

The compressed dosage form as disclosed may be characterized in that compressed dosage forms of the same size, form and composition are showing, with and without curing at 40° C. for 24 hours, a similarity factor f₂ of 50 or more of their compared active ingredient release profiles from a drug dissolution test at pH 6.8.

The compressed dosage form as disclosed may be characterized in that compressed dosage forms of same size, form and composition are showing, with and without storing in HDPE containers at 40° C. and 75% relative humidity for one month, a similarity factor f₂ of 50 or more of the compared active ingredient release profiles from a drug dissolution test at pH 6.8.

The compressed dosage form as disclosed may show an active ingredient release of 60% or more within 24 hours in a dissolution test at pH 6.8 according to USP (for instance USP 31).

### F2-Value

The f2-value is known to the skilled person from the requirements for bioequivalence studies as defined by the Food and Drug Administration (FDA) of the United States of America. These values are, for example, available in documents like “Guidance for Industry; Waiver of In Vivo Bioavailability and Bioequivalence Studies for Immediate-Release Solid Oral Dosage Forms Based on Biopharmaceutics Classification System” (U.S. Department of Health and Human Services, Food and Drug Administration, Center for Drug Evaluation and Research (CDER), August 2000) or from other versions of this document, or from other documents or guidelines from the FDA or CDER concerning bioavailability and bioequivalence Studies. All these documents are available in the Internet and well known to the skilled person in the field of pharmacy and galenics. In the above-mentioned document from August 2000 the calculation of the similarity factor (f2) is defined on p. 7:

When comparing the test and reference products, dissolution profiles should be compared using a similarity factor (f2). The similarity factor is a logarithmic reciprocal square root transformation of the sum of squared error and is a measurement of the similarity in the percent (%) of dissolution between the two curves.

f2=50·log{[1+(1/n)Σₜ₌₁n(Rₜ−Tₜ)²]^{-0.5}·100}

Two dissolution profiles are considered similar when the f2 value is ≥50.

### Biologically Active Ingredient

The biologically active ingredient is preferably a pharmaceutically active ingredient and/or a nutraceutically active ingredient.

The one or more biologically active ingredient(s) may be selected from the groups of analgesics, antibiotics or anti-infectives, antibodies, antiepileptics, antigens from plants, antirheumatics, benzimidazole derivatives, beta-blocker, cardiovascular drugs, chemotherapeutics, CNS drugs, digitalis glycosides, gastrointestinal drugs, e.g. proton pump inhibitors, enzymes, hormones, liquid or solid natural extracts, oligonucleotides, peptide hormones proteins, therapeutic bacteria, peptides, proteins and their (metal)salts, i.e. aspartates, chlorides, orthates, urology drugs and vaccines. Further examples of biologically active ingredient that may be are for instance acamprosat, aescin, amylase, acetylsalicylic acid, adrenalin, 5-amino salicylic acid, aureomycin, bacitracin, balsalazine, beta carotene, bicalutamid, bisacodyl, bromelain, bromelain, budesonide, calcitonin, carbamacipine, carboplatin, cephalosporins, cetrorelix, clarithromycin, chloromycetin, cimetidine, cisapride, cladribine, clorazepate, cromalyn, 1-deaminocysteine-8-D-arginine-vasopressin, deramciclane, detirelix, dexiansoprazole, diclofenac, didanosine, digitoxin and other digitalis glycosides, dihydrostreptomycin, dimethicone, divalproex, drospirenone, duloxetine, enzymes, erythromycin, esomeprazole, estrogens, etoposide, famotidine, fluorides, garlic oil, glucagon, granulocyte colony stimulating factor (G-CSF), heparin, hydrocortisone, human growth hormon (hGH), ibuprofen, ilaprazole, insulin, Interferon, Interleukin, Intron A, ketoprofen, lansoprazole, leuprolidacetat lipase, lipoic acid, lithium, kinin, memantine, mesalazine, methenamine, milameline, minerals, minoprazole, naproxen, natamycin, nitrofurantion, novobiocin, olsalazine, omeprazole, orothates, pancreatin, pantoprazole, parathyroidhormone, paroxetine, penicillin, perprazol, pindolol, polymyxin, potassium, pravastatin, prednisone, preglumetacin progabide, pro-somatostatin, protease, quinapril, rabeprazole, ranitidine, ranolazine, reboxetine, rutosid, somatostatin streptomycin, subtilin, sulfasalazine, sulphanilamide, tamsulosin, tenatoprazole, trypsin, valproic acid, vasopressin, vitamins, zinc, including salts, derivatives, polymorphs, isomorphs, or any kinds of mixtures or combinations thereof.

It is evident to a skilled person that there is a broad overlap between the terms pharmaceutically and nutraceutically active ingredients, excipients and compositions respectively a pharmaceutical or a nutraceutical dosage form. Many substances listed as nutraceuticals may also be used as pharmaceutically active ingredients. Depending on the specific application and local authority legislation and classification, the same substance may be listed as a pharmaceutically or a nutraceutically active ingredient respectively a pharmaceutical or a nutraceutical composition or even both.

Nutraceuticals are well known to the skilled person. Nutraceuticals are often defined as extracts of foods claimed to have medical effects on human health. Thus, nutraceutically active ingredients may display pharmaceutical activities as well: Examples for nutraceutically active ingredients may be resveratrol from grape products as an antioxidant, soluble dietary fiber products, such as psyllium seed husk for reducing hypercholesterolemia, broccoli (sulphane) as a cancer preventive and soy or clover (isoflavonoids) to improve arterial health. Thus, it is clear that many substances listed as nutraceuticals may also be used as pharmaceutically active ingredients.

Typical nutraceuticals or nutraceutically active ingredients may include probiotics and prebiotics. Probiotics are living microorganisms believed to support human or animal health when consumed. Prebiotics are nutraceuticals or nutraceutically active ingredients that induce or promote the growth or activity of beneficial microorganisms in the human or animal intestine.

Examples for nutraceuticals are resveratrol from grape products, omega-3-fatty acids or pro-anthocyanines e.g. from bilberries, blueberries or black currants as antioxidants, soluble dietary fiber products, such as psyllium seed husk for reducing hypercholesterolemia, broccoli (sulphane) as a cancer preservative, and soy or clover (isoflavonoids) to improve arterial health. Other nutraceuticals examples are flavonoids, antioxidants, alpha-linoleic acid from flax seed, beta-carotene from marigold petals or antocyanins from various sources. Sometimes the expression neutraceuticals or nutriceuticals are used as synonyms for nutraceuticals. A preferred biologically active ingredient is for instance metoprolol.

### Pharmaceutical or Nutraceutical Excipients

Pharmaceutical or nutraceutical excipients may be selected from the groups of antioxidants, brighteners, binding agents, cushioning agents, flavoring agents, flow aids, glidants, penetration-promoting agents, pigments, plasticizers, excipient polymers (different from the polymer mixture A or from the water-soluble cellulose B, for instance polymers such as microcrystalline cellulose or PVP), pore-forming agents and stabilizers or any combinations thereof.

Preferably, the pharmaceutical or nutraceutical excipients may comprise microcystalline cellulose, glycerol monostearate, lactose, silica, Mg-stearate, croscarmellose sodium and/or sodium stearyl fumarate.

### EXAMPLES

### Polymers Used in the Examples

EUDRAGIT® NM 30 D is a commercially available 30% by weight aqueous dispersion of a copolymer comprising polymerized units of about 70% by weight of ethyl acrylate and 30% by weight of methyl methacrylate.

EUDRAGIT® L 100-55 is a commercially available copolymer comprising polymerized units of 50% by weight of ethyl acrylate and 50% by weight of methacrylic acid.

EUDRAGIT® L 30 D-55 is a commercially available 30% by weight aqueous dispersion of a copolymer comprising polymerized units of 50% by weight of ethyl acrylate and 50% by weight of methacrylic acid.

EUDRAGIT® FL 30 D-55 (Evonik Nutrition & Care GmbH, Darmstadt, Germany) is a commercially available 30% by weight aqueous dispersion of a copolymer from a two-stage emulsion polymerization process, with a core of about 75% by weight, comprising polymerized units of about 70% by weight of ethyl acrylate and 30% by weight of methyl methacrylate, and a shell of about 25% by weight, comprising polymerized units of 50% by weight of ethyl acrylate and 50% by weight of methacrylic acid.

EUDRAGIT® S 100 is a commercially available copolymer comprising polymerized units of 70% by weight of methyl methacrylate and 30% by weight of methacrylic acid.

EUDRAGIT® RL 30 D-55 is a commercially available 30% by weight aqueous dispersion of a copolymer comprising polymerized units of about 30% by weight of ethyl acrylate, 60% by weight of methyl methacrylate and 10% trimethylammoniumethyl methacrylate. EUDRAGIT® RL PO is a copolymer in powder form comprising polymerized units of about 30% by weight of ethyl acrylate, 60% by weight of methyl methacrylate and 10% by weight of trimethylammoniumethyl methacrylate.

EUDRAGIT® E PO is a copolymer in powder form comprising polymerized units of about 25% by weight of butyl methacrylate, 25% by weight of methyl methacrylate and 50% by weight of dimethylaminoethyl methacrylate.

Water-soluble celluloses as used are hydroxyethyl cellulose (HEC), hydroxypropyl cellulose (HPC-LM) and hydroxypropyl methyl cellulose (HPMC K4M and 6CPS). Ethyl cellulose (EC) is a water-insoluble cellulose. PVP is polyvinyl pyrrolidone, PVA is polyvinyl alcohol.

### 1. Experiments with EUDRAGIT® FL 30 D-65 and Cellulosic Polymer Combinations

### 1.1 Formulations

### 1.1.1 Formulations of Examples I-1 to I-8 (According to the Invention)

TABLE 1
Compositions (%/w/w)

| | | | | | | | | |
|---|---|---|---|---|---|---|---|---|
| Experiment number | I-1 | I-2 | I-3 | I-4 | I-5 | I-6 | I-7 | I-8 |

Co-processing step

| | | | | | | | | |
|---|---|---|---|---|---|---|---|---|
| Method of co- | SD | SD | SD | SD | SD | SD | SD | SD |
| processing # | | | | | | | | |
| EUDRAGIT ® | 34.39* | 34.39* | 30.57* | 26.75* | 26.75* | 34.39* | 34.39* | 26.75* |
| FL 30 D-55 (FL) | | | | | | | | |
| HPMC K4M | 3.82* | — | — | — | — | — | — | — |
| HPMC 6CPS | — | 3.82* | 7.64 * | 11.46* | — | — | — | — |
| EUDRAGIT ® | — | — | — | — | — | — | — | — |
| NM 30 D (NM) | | | | | | | | |
| EUDRAGIT ® | — | — | — | — | — | — | — | — |
| L 30 D-55 (L) | | | | | | | | |
| EUDRAGIT ® | — | — | — | — | — | — | — | — |
| L 100-55 (L) | | | | | | | | |
| HPC-LM | — | — | — | — | — | — | 3.82* | 11.46* |
| HEC | — | — | — | — | 11.46* | 3.82* | — | — |
| EC | — | — | — | — | — | — | — | — |
| Water (q.s to % w/w | q.s. to | q.s. to | q.s. to | q.s.to | q.s.to | q.s.to | q.s.to | q.s. to |
| solids) | 5% | 10% | 10% | 10% | 10% | 10% | 10% | 10% |

Tablet compression step

| | | | | | | | | |
|---|---|---|---|---|---|---|---|---|
| Metoprolol Succinate | 15.29 | 15.29 | 15.29 | 15.29 | 15.29 | 15.29 | 15.29 | 15.29 |
| MCC PH 102 | 45.85 | 45.85 | 45.85 | 45.85 | 45.85 | 45.85 | 45.85 | 45.85 |
| Aerosil ® 200 | 0.31 | 0.31 | 0.31 | 0.31 | 0.31 | 0.31 | 0.31 | 0.31 |
| Magnesium Stearate | 0.34 | 0.34 | 0.34 | 0.34 | 0.34 | 0.34 | 0.34 | 0.34 |
| Total | 100 | 100 | 100 | 100 | 100 | 100 | 100 | 100 |
| Ratio of Polymers | 90:10 | 90:10 | 80:20 | 70:30 | 70:30 | 90:10 | 90:10 | 70:30 |
| FL:Cellulose | | | | | | | | |
| Ratio of Polymers | — | — | — | — | — | — | — | — |
| NM:L:Cellulose | | | | | | | | |

# For each batch, ingredients marked with * were co-processed together in a single spray drying step.
Abbreviations:
FD = Freeze drying; SD = Spray drying; PM = Physical mixing; HPMC = Hydroxypropyl Methyl Cellulose; HPC = Hydroxypropyl Cellulose; HEC = Hydroxyethyl Cellulose; EC = Ethyl cellulose; API = Active Pharmaceutical Ingredient; MCC = Microcrystalline Cellulose

### 1.1.2 Formulations of Examples I-9 to I-16 (According to the Invention)

TABLE 1
(continued): Compositions (%/w/w)

| | | | | | | | | |
|---|---|---|---|---|---|---|---|---|
| Experiment number | I-9 | I-10 | I-11 | I-12 | I-13 | I-14 | I-15 | I-16 |

Co-processing step

| | | | | | | | | |
|---|---|---|---|---|---|---|---|---|
| Method of co- | SD | SD + | FD | FD | FD | SD + | SD + | SD + |
| processing # | | PM | | | | PM | PM | PM |
| EUDRAGIT ® | — | — | — | 34.39** | 30.57** | 34.39* | 35* | 35* |
| FL 30 D-55 (FL) | | | | | | | | |
| HPMC K4M | — | — | — | — | — | — | — | — |
| HPMC 6CPS | 7.64* | 7.64* | 7.64** | 3.82** | 7.64** | 3.82*** | — | — |
| EUDRAGIT ® | 22.93* | 22.93* | 22.93** | — | — | — | — | — |
| NM 30 D (NM) | | | | | | | | |
| EUDRAGIT ® | 7.64* | — | 7.64* | — | — | — | — | — |
| L 30 D-55 (L) | | | | | | | | |
| EUDRAGIT ® | — | 7.64*** | — | — | — | — | — | — |
| L 100-55 (L) | | | | | | | | |
| HPC-LM | — | — | — | — | — | — | 15*** | — |
| HEC | — | — | — | — | — | — | — | 15 *** |
| EC | — | — | — | — | — | — | — | — |
| Water (q.s to % w/w | q.s. to | q.s. to | q.s to | q.s. to | q.s to | q.s. to | q.s. to | q.s. to |
| solids) | 10% | 10% | 30% | 30% | 30% | 10% | 10% | 10% |

Tablet compression step

| | | | | | | | | |
|---|---|---|---|---|---|---|---|---|
| Metoprolol Succinate | 15.29 | 15.29 | 15.29 | 15.29 | 15.29 | 15.29 | 15.38 | — |
| Theophylline | — | — | — | — | — | — | — | 15.29 |
| MCC PH 102 | 45.85 | 45.85 | 45.85 | 45.85 | 45.85 | 45.85 | 33.10 | 45.85 |
| Aerosil ® 200 | 0.31 | 0.31 | 0.31 | 0.31 | 0.31 | 0.31 | 0.58 | 0.31 |
| Magnesium Stearate | 0.34 | 0.34 | 0.34 | 0.34 | 0.34 | 0.34 | 0.92 | 0.92 |
| Total | 100 | 100 | 100 | 100 | 100 | 100 | 100 | 100 |
| Ratio of Polymers | — | — | — | 90:10 | 80:20 | 90:10 | 70:30 | 70:30 |
| FL:Cellulose | | | | | | | | |
| Ratio of Polymers | 60:20:20 | 60:20:20 | 60:20:20 | — | — | — | — | — |
| NM:L:Cellulose | | | | | | | | |

# For each batch, ingredients marked with * were co-processed together in a single spray drying step. For each batch, ingredients marked with ** were co-processed together in a single freeze-drying step. Ingredients marked with *** were co-processed by only physically mixing with the spray dried powder in the same batch.
Abbreviations:
FD = Freeze drying; SD = Spray drying; PM = Physical mixing; HPMC = Hydroxypropyl Methyl Cellulose; HPC = Hydroxypropyl Cellulose; HEC = Hydroxyethyl Cellulose; EC = Ethyl cellulose; API = Active Pharmaceutical Ingredient; MCC = Microcrystalline Cellulose

### 1.1.3 Formulations of Examples I-17 to I-20 (According to the Invention)

TABLE 1
(continued): Compositions (%/w/w)

| | | | | |
|---|---|---|---|---|
| Experiment number | I-17 | I-18 | I-19 | I-20 |

Co-processing step

| | | | | |
|---|---|---|---|---|
| Method of co-processing # | SD | SD | SD | SD |
| EUDRAGIT ® | 34.39* | 34.39* | 34.39* | 34.39* |
| FL 30 D-55 (FL) | | | | |
| HPMC 6CPS | 3.82* | 3.82* | 3.82* | 3.82* |
| Water (q.s to % w/w solids) | q.s. to 10% | q.s. to 10% | q.s. to 10 | q.s. to 10% |

Tablet compression step

| | | | | |
|---|---|---|---|---|
| Mesalamine | 15.29 | — | — | — |
| Niacin | — | 15.29 | — | — |
| Metformin Hydrochloride | — | — | 15.29 | — |
| Theophylline | — | — | — | 15.29 |
| MCC PH 102 | 45.85 | 45.85 | 45.85 | 45.85 |
| Aerosil ® 200 | 0.31 | 0.31 | 0.31 | 0.31 |
| Magnesium Stearate | 0.34 | 0.34 | 0.34 | 0.34 |
| Total | 100 | 100 | 100 | 100 |
| Ratio of Polymers | 80:20 | 80:20 | 80:20 | 80:20 |
| FL:Cellulose | | | | |

# For each batch, ingredients marked with * were co-processed together in a single spray drying step.
Abbreviations:
FD = Freeze drying; SD = Spray drying; PM = Physical mixing; HPMC = Hydroxypropyl Methyl Cellulose; HPC = Hydroxypropyl Cellulose; HEC = Hydroxyethyl Cellulose; EC = Ethyl cellulose; API = Active Pharmaceutical Ingredient; MCC = Microcrystalline Cellulose

### 1.1.4 Formulations of Examples C-1 to C-7 (Comparative)

TABLE 1
(continued): Compositions (%/w/w)

| | | | | | | | |
|---|---|---|---|---|---|---|---|
| Experiment number | C-1 | C-2 | C-3 | C-4 | C-5 | C-6 | C-7 |

Co-processing step

| | | | | | | | |
|---|---|---|---|---|---|---|---|
| Method of co-processing # | SD | SD | SD | SD | SD | SD | SD |
| EUDRAGIT ® | 50.40 | 30.57* | — | 50.40* | 50.40* | 50.40* | 50.40* |
| FL 30 D-55 (FL) | | | | | | | |
| HPMC K4M | — | — | — | — | — | — | — |
| HPMC 6CPS | — | — | — | — | — | — | — |
| EUDRAGIT ® | — | — | 22.93* | — | — | — | — |
| NM 30 D (NM) | | | | | | | |
| EUDRAGIT ® | — | — | 7.64* | — | — | — | — |
| L 30 D-55 (L) | | | | | | | |
| EUDRAGIT ® | — | — | — | — | — | — | — |
| L 100-55 (L) | | | | | | | |
| HPC-LM | — | — | — | — | — | — | — |
| HEC | — | — | — | — | — | — | — |
| EC | — | 7.64* | 7.64* | — | — | — | — |
| Water (q.s to % w/w solids) | q.s to | q.s.to | q.s. to | q.s.to | q.s.to | q.s.to | q.s.to |
| | 15%. | 10% | 10% | 10% | 10% | 10% | 10% |

Tablet compression step

| | | | | | | | |
|---|---|---|---|---|---|---|---|
| Metoprolol Succinate | 21.73 | 15.29 | 15.29 | — | — | — | — |
| Mesalamine | — | — | — | 21.73 | — | — | — |
| Niacin | — | — | — | — | 21.73 | — | — |
| Metformin Hydrochloride | — | — | — | — | — | 21.73 | — |
| Theophylline | — | — | — | — | — | — | 21.73 |
| MCC PH 102 | — | 45.85 | 45.85 | 26.30 | 26.30 | 26.30 | 26.30 |
| Aerosil ® 200 | 26.30 | 0.31 | 0.31 | 0.50 | 0.50 | 0.50 | 0.50 |
| Magnesium Stearate | 0.50 | 0.34 | 0.34 | 1.00 | 1.00 | 1.00 | 1.00 |
| Total | 100 | 100 | 100 | 100 | 100 | 100 | 100 |
| Ratio of Polymers | 100:0 | 80:20 | — | 100:0 | 100:0 | 100:0 | 100:0 |
| FL:Cellulose | | | | | | | |
| Ratio of Polymers | — | — | 60:20:20 | — | — | — | — |
| NM:L:Cellulose | | | | | | | |

# For each batch, ingredients marked with * were co-processed together in a single spray drying step.
Abbreviations:
FD = Freeze drying; SD = Spray drying; PM = Physical mixing; HPMC = Hydroxypropyl Methyl Cellulose; HPC = Hydroxypropyl Cellulose; HEC = Hydroxyethyl Cellulose; EC = Ethyl cellulose; API = Active Pharmaceutical Ingredient; MCC = Microcrystalline Cellulose

### 1.2 Process Details for Examples C-1 to C-7 & I-1 to I-8 & I-17 to I-20

### a) Spray Drying Step:

Cellulose component, if any in the formulation, was added slowly in water under continuous stirring to form a colloidal dispersion.
EUDRAGIT® FL 30 D-55 was added slowly to the colloidal dispersion from above step.
The dispersion was then passed thru 60 #ASTM sieve (250 μm) and then it was used for spray drying using lab scale Buchi spray drier.

Process Parameters for Spray Drying:

| | | C-2, I-1 to I-8 |
|---|---|---|
| Experiment number | C-1 and C-4 to C-7 | & I-17 to I-20 |
| Inlet Temperature (° C.) | 50-55 | 65-75 |
| Aspirator (%) | 90-95 | 90-95 |

### b) Tablet Compression Step:

All the ingredients were passed thru 30 #ASTM sieve (590 μm).
The spray dried polymeric powder from above was mixed geometrically with the API and other tableting excipients (except magnesium stearate) as mentioned in Table 1.
Magnesium stearate was added at the end after passing it thru 80 #ASTM sieve (180 μm) to the blend from above step and mixed uniformly.
The blend was compressed into tablets using D tooling fitted on a rotary compression machine.

Process Parameters for Tablet Compression:

| | | C-2, I-1 to I-8 |
|---|---|---|
| Experiment number | C-1 and C-4 to C-7 | & I-17 to I-20 |
| Punch size (mm) | 9 | 12.5 |
| Weight (mg) | 230 | 650-655 |
| Hardness (N) | 80-90 | 70-90 |

### 1.3 Process Details for Examples I-9, I-10 and C-3

### a) Spray Drying Step:

Cellulose component was added slowly in water under continuous stirring to form a colloidal dispersion.
EUDRAGIT® NM 30 D was added slowly to EUDRAGIT® L 30 D-55 (if any) under stirring to mix together.
EUDRAGIT® dispersion from above step was added to the colloidal cellulose dispersion under stirring.
The dispersion was then passed thru 60 #ASTM sieve (250 μm) and then it was used for spray drying using lab scale Buchi spray drier.

Process Parameters for Spray Drying:

| Experiment number | I-9, I-10 & C-3 |
|---|---|
| Inlet Temperature (° C.) | 40-50 |
| Aspirator (%) | 90-95 |

### b) Tablet Compression Step:

All the ingredients were passed thru 30 #ASTM sieve (590 μm).
The spray dried polymeric powder from above was mixed geometrically with the API and other tableting excipients (except magnesium stearate) as mentioned in Table 1. For experiment I-10, EUDRAGIT® L 100 powder was mixed uniformly with the spray dried powder before mixing it with other ingredients.
Magnesium stearate was added at the end after passing it thru 80 #ASTM sieve (180 μm) to the blend from above step and mixed uniformly.
The blend was compressed into tablets using D tooling fitted on a rotary compression machine.

Process Parameters for Tablet Compression:

| Experiment number | I-9, 1-10 & C-3 |
|---|---|
| Punch size (mm) | 12.5 |
| Weight (mg) | 650-655 |
| Hardness (N) | 70-90 |

### 1.4 Process Details for Examples I-11 to I-13

### a) Freeze Drying Step:

Cellulose component, if any in the formulation, was added slowly in water under continuous stirring to form a colloidal dispersion.
EUDRAGIT® NM 30 D was added slowly to EUDRAGIT® L 30 D-55 (if any) under stirring to mix together.
EUDRAGIT® dispersion from above step was added to the colloidal cellulose dispersion under stirring.
The dispersion was then passed thru 60 #ASTM sieve (250 μm) and then it was used for freeze drying using lab scale Vertis freeze drier.

### Process Parameters for Freeze Drying:

| Thermal treatment | Temperature: −40° C. | | | |
|---|---|---|---|---|
| phase | Time: 90 min | | | |
| Freeze, | Freezing temp: −30° C. | | | |
| | Additional Time: 60 min | | | |
| Condenser, | Condenser: −40° C. | | | |
| Vacuum phase | Vacuum: 400 mTorr | | | |
| | 35-40° C. | | | |
| | | | | Vacuum |
| Drying cycle step | Step | Temp (° C.) | Time (min) | (mTorr) |
| | 1 | −30 | 120 | 400 |
| | 2 | −15 | 120 | 400 |
| | 3 | 0 | 240 | 400 |
| | 4 | +15 | 180 | 400 |
| | 5 | +25 | 180 | 400 |

| Secondary drying | Secondary set point: +40° C. | | |
|---|---|---|---|
| Post heat setting | Temperature: | Time: | Vacuum: |
| | +25° C. | 600 min | 400 mTorr |

### b) Tablet Compression Step:

All the ingredients were passed thru 30 #ASTM sieve (590 μm).
The spray dried polymeric powder from above was mixed geometrically with the API and other tableting excipients (except magnesium stearate) as mentioned in Table 1.
Magnesium stearate was added at the end after passing it thru 80 #ASTM sieve (180 μm) to the blend from above step and mixed uniformly.
The blend was compressed into tablets using D tooling fitted on a rotary compression machine.

### Process Parameters for Tablet Compression:

| Experiment number | I-11 to I-13 |
|---|---|
| Punch size (mm) | 12.5 |
| Weight (mg) | 650-655 |
| Hardness (N) | 70-90 |

### 1.5 Process Details for Experiments I-14 to I-16

### a) Spray Drying and Physical Mixing Step:

EUDRAGIT® FL 30 D was diluted with water to the solids content of 15% (w/w).
The dispersion was then passed thru 60 #ASTM sieve (250 μm) and then it was used for spray drying using lab scale Buchi spray drier.

### Process Parameters for Spray Drying:

| Experiment number | I-14 to I-16 |
|---|---|
| Inlet Temperature (° C.) | 50-55 |
| Aspirator (%) | 90-95 |

The spray dried polymeric powder from above step was mixed uniformly with celluloses and passed thru 30 #ASTM sieve (590 μm)

### b) Tablet Compression Step:

All the ingredients were passed thru 30 #ASTM sieve (590 μm).
The spray dried and physically mixed polymeric powder from above was mixed geometrically with the API and other tableting excipients (except magnesium stearate) as mentioned in Table 1.
Magnesium stearate was added at the end after passing it thru 80 #ASTM sieve (180 μm) to the blend from above step and mixed uniformly.
The blend was compressed into tablets using D tooling fitted on a rotary compression machine.

### Process Parameters for Tablet Compression:

| Experiment number | I-14 to I-16 |
|---|---|
| Punch size (mm) | 12.5 |
| Weight (mg) | 650-655 |
| Hardness (N) | 70-90 |

### 1.6 Analysis of the Compressed Tablets

a) Dissolution: All the samples were studied for dissolution profile (24 hrs) using recommended dissolution equipment and media.
b) Curing study: All the compressed tablet samples were studied for curing effect by exposing it to 40-50° C. for 24 hrs. Dissolution profiles before and after curing were compared using f2 value.
c) Stability: The uncured samples were packed in HDPE containers and charged in a stability chamber maintained at 40° C./75% RH. The samples were analyzed at least after 1 month of storage and studied for any change in dissolution profile using 12 test.

### 1.7 a) Results for the Examples According to the Invention (I-1 to I-20)

| | Experiment numbers | | | | | | | |
|---|---|---|---|---|---|---|---|---|
| Parameters | I-1 | I-2 | I-3 | I-4 | I-5 | I-6 | I-7 | I-8 |
| No Curing effect | 63 | 76.5 | 97.6 | 77.9 | 65.9 | 82.1 | 66 | 93.2 |
| on dissolution | | | | | | | | |
| profile | | | | | | | | |
| Acceptance | | | | | | | | |
| criteria: | | | | | | | | |
| f2 > 50 | | | | | | | | |
| 1-month Stability | 74.3 | 94.5 | 78.8 | 74.9 | 73.7 | 78.0 | 83.1 | 90.5 |
| Acceptance | | | | | | | | |
| criteria: | | | | | | | | |
| f2 > 50 | | | | | | | | |
| Dissolution | 79.65 | 89.62 | 96.11 | 102.9 | 107 | 92.83 | 92.01 | 89.88 |
| profile | | | | | | | | |
| Acceptance | | | | | | | | |
| criteria: | | | | | | | | |
| NLT 60% in 24 | | | | | | | | |
| hrs, pH 6.8 | | | | | | | | |
| Batch passing all | Yes | Yes | Yes | Yes | Yes | Yes | Yes | Yes |
| the acceptance | | | | | | | | |
| criteria | | | | | | | | |
| | Experiment numbers | | | | | | | |
| Parameters | I-9 | I-10 | I-11 | I-12 | I-13 | I-14 | I-15 | I-16 |
| No Curing effect | 93.1 | 93.6 | 78.4 | 87.9 | 83.6 | 66 | 88.2 | 93.3 |
| on dissolution | | | | | | | | |
| profile | | | | | | | | |
| Acceptance | | | | | | | | |
| criteria: | | | | | | | | |
| f2 > 50 | | | | | | | | |
| 1-month Stability | 64.5 | 85.8 | 76.9 | 50.5 | 83.6 | 74.1 | 74.5 | 95.6 |
| Acceptance | | | | | | | | |
| criteria: | | | | | | | | |
| f2 > 50 | | | | | | | | |
| Dissolution | 97.01 | 100.65 | 89.91 | 98.62 | 77.83 | 96.53 | 92.19 | 100.49 |
| profile | | | | | | | | |
| Acceptance | | | | | | | | |
| criteria: | | | | | | | | |
| NLT 60% in 24 | | | | | | | | |
| hrs, pH 6.8 | | | | | | | | |
| Batch passing all | Yes | Yes | Yes | Yes | Yes | Yes | Yes | Yes |
| the acceptance | | | | | | | | |
| criteria | | | | | | | | |
| | | | | | Experiment numbers | | | |
| | | | | Parameters | I-17 | I-18 | I-19 | I-20 |
| | | | | No Curing effect | 90.3 | 85.0 | 73.8 | 78.0 |
| | | | | on dissolution | | | | |
| | | | | profile | | | | |
| | | | | Acceptance | | | | |
| | | | | criteria: | | | | |
| | | | | f2 > 50 | | | | |
| | | | | 1-month | 82.4 | 69.9 | 83.5 | 96.3 |
| | | | | Stability | | | | |
| | | | | Acceptance | | | | |
| | | | | criteria: | | | | |
| | | | | f2 > 50 | | | | |
| | | | | Dissolution | 62 | 99.2 | 103.58 | 79.03 |
| | | | | profile | | | | |
| | | | | Acceptance | | | | |
| | | | | criteria: | | | | |
| | | | | NLT 60% in 24 | | | | |
| | | | | hrs, pH 6.8 | | | | |
| | | | | Batch passing | Yes | Yes | Yes | Yes |
| | | | | all the | | | | |
| | | | | acceptance | | | | |
| | | | | criteria | | | | |

### 1.7 b) Results for the Comparative Examples (C-1 to C-7)

| | Experiment numbers | | | | | | |
|---|---|---|---|---|---|---|---|
| Parameters | C-1 | C-2 | C-3 | C-4 | C-5 | C-6 | C-7 |
| No Curing effect | <50 | 87.9 | 65.6 | 46.3 | 29.0 | 47.6 | 60.5 |
| on dissolution | | | | | | | |
| profile | | | | | | | |
| Acceptance | | | | | | | |
| criteria: | | | | | | | |
| f2 > 50 | | | | | | | |
| 1-month | — | 33.3 | 29.5 | Not done | Not done | Not done | 47 |
| Stability | | | | | | | |
| Acceptance | | | | | | | |
| criteria: | | | | | | | |
| f2 > 50 | | | | | | | |
| Dissolution | 101 | 101.46 | 104 | 96.55 | 100 | 100 | 98.73 |
| profile | | | | | | | |
| Acceptance | | | | | | | |
| criteria: | | | | | | | |
| NLT 60% in 24 | | | | | | | |
| hrs, pH 6.8 | | | | | | | |
| Batch passing | No | No | No | No | No | No | No |
| all the | | | | | | | |
| acceptance | | | | | | | |
| criteria | | | | | | | |

### 1.8 Important Findings from Above Experiments

a) EUDRAGIT® FL 30 D-55 (powdered form) when used alone for direct compression, fails to give curing free dissolution profiles.
b) EUDRAGIT® FL 30 D-55 when combined with water soluble celluloses gives the desired curing free, stable and dissolution properties.
c) EUDRAGIT® FL 30 D-55 when combined with water insoluble celluloses (example—Ethyl cellulose) gives the desired curing free profiles but fails to maintain dissolution profile on stability, wherein the f2 value falls below 50 within 1 month of storage.

### 2. Experiments with EUDRAGIT® FL 30 D-55 and Non-Cellulosic Polymer Combinations

### 2.1 Formulations

### 2.1.1 Formulations of Examples C-8 to C-14 (Comparative)

TABLE 1
(continued): Compositions (%/w/w)

| | | | | | | | |
|---|---|---|---|---|---|---|---|
| Experiment number | C-8 | C-9 | C-10 | C-11 | C-12 | C-13 | C-14 |

Co-processing step

| | | | | | | | |
|---|---|---|---|---|---|---|---|
| Method of co-processing# | SD | SD | SD + | SD + | SD | FD | SD + |

| | | | | | | | |
|---|---|---|---|---|---|---|---|
| | | | PM | PM | | | PM |
| EUDRAGIT ® | 34.39* | 34.39* | 34.39* | 30.57* | 34.39* | 34.39* | 34.39* |
| FL 30 D-55 (FL) | | | | | | | |
| PVP K30 | 3.82* | NA | — | NA | — | — | — |
| PVP K25 | — | — | 3.82* | — | — | — | — |
| PVA | NA | 3.82* | — | 3.82*** | — | — | — |
| EUDRAGIT ® | — | — | — | — | 3.82* | — | — |
| RL 30 D | | | | | | | |
| EUDRAGIT ® | — | — | — | — | — | 3.82** | — |
| E PO | | | | | | | |
| EUDRAGIT ® | — | — | — | — | — | — | 3.82* |
| S 100 | | | | | | | |
| Water | q.s: to | q.s: to | q.s: to | q.s: to | q.s: to | q.s: to | q.s: to |
| | 10% | 10% | 10% | 10% | 10% | 30% | 10% |
| | w/w | w/w | w/w | w/w | w/w | w/w | w/w |
| | solids | solids | solids | solids | solids | solids | solids |

Tablet compression step

| | | | | | | | |
|---|---|---|---|---|---|---|---|
| API (Metoprolol | 15.29 | 15.29 | 15.29 | 15.29 | 15.29 | 15.29 | 15.29 |
| succinate) | | | | | | | |
| MCC PH 102 | 45.85 | 45.85 | 45.85 | 45.85 | 45.85 | 45.85 | 45.85 |
| Aerosil 200 | 0.306 | 0.306 | 0.306 | 0.306 | 0.306 | 0.306 | 0.306 |
| Magnesium Stearate | 0.343 | 0.343 | 0.343 | 0.343 | 0.343 | 0.343 | 0.343 |
| Total | 100 | 100 | 100 | 100 | 100 | 100 | 100 |
| Ratio of Polymer | 90:10 | 90:10 | 90:10 | 90:10 | 90:10 | 90:10 | 90:10 |
| FL:Non-Celluloses | | | | | | | |
| Ratio of Polymer | 75:25 | 75:25 | 75:25 | 75:25 | 75:25 | 75:25 | 75:25 |
| EUDRAGIT ® (NM:L) | | | | | | | |

#For each batch, ingredients marked with * were co-processed together in a single spray drying step. For each batch, ingredients marked with ** were co-processed together in a single freeze drying step. For each batch, ingredients marked with *** were co-processed together by only physically mixing together.
Abbreviations: FD = Freeze drying; SD = Spray drying; PM = Physical mixing; API = Active Pharmaceutical Ingredient; MCC = Microcrystalline Cellulose; PVP = Polyvinyl Pyrrolidone; PVA = Polyvinyl Acetate

### 2.2 Process Details for Experiments C-8, C-9 and C-12

### a) Spray Drying Step:

Non-Cellulosic component apart from EUDRAGIT®, if any in the formulation, was added slowly in water under continuous stirring to form a colloidal dispersion.
EUDRAGIT® FL 30 D-55 was added slowly to the colloidal dispersion from above step.
Other EUDRAGIT® dispersion were prepared separately and to it EUDRAGIT® FL 30 D-55 was added.
The dispersion was then passed thru 60 #ASTM sieve (250 μm) and then it was used for spray drying using lab scale Buchi spray drier.

Process Parameters for Spray Drying:

| Experiment number | C-8, C-9 & C-12 |
|---|---|
| Inlet Temperature (° C.) | 70-73 |
| Aspirator (%) | 85-90 |

### b) Tablet Compression Step:

All the ingredients were passed thru 30 #ASTM sieve (590 μm).
The spray dried polymeric powder from above was mixed geometrically with the API and other tableting excipients (except magnesium stearate) as mentioned in Table 1.
Magnesium stearate was added at the end after passing it thru 80 #ASTM sieve (180 μm) to the blend from above step and mixed uniformly.
The blend was compressed into tablets using D tooling fitted on a rotary compression machine.

Process Parameters for Tablet Compression:

| Experiment number | C-8 | C-9 | C-12 |
|---|---|---|---|
| Punch size (mm) | 9 | 12.5 | 12.5 |

| | | |
|---|---|---|
| Weight (mg) | 230 | 650-655 |

| | | | |
|---|---|---|---|
| Hardness (N) | 80-90 | 70-90 | 70-90 |

### 2.3 Process Details for Experiments C-13

### a) Freeze Drying Step:

EUDRAGIT® FL 30 D dispersion was prepared and it was kept under stirring.
Other EUDRAGIT® mentioned in the experiments its dispersion was prepared separately.
EUDRAGIT® dispersion from above step was added to the EUDRAGIT® FL 30 D dispersion under stirring.
The dispersion was then passed thru 60 #ASTM sieve (250 μm) and then it was used for freeze drying using lab scale Vertis freeze drier.

Process Parameters for Freeze Drying:

| Thermal treatment | Temperature: −40° C. | | | |
|---|---|---|---|---|
| phase | Time: 90 min | | | |
| Freeze, | Freezing temp: −30° C. | | | |
| | Additional Time: 60 min | | | |
| Condenser, | Condenser: −40° C. | | | |
| Vacuum phase | Vacuum: 400 mTorr | | | |
| | 35-40° C. | | | |
| | | | | Vacuum |
| Drying cycle step | Step | Temp (° C.) | Time (min) | (mTorr) |
| | 1 | −30 | 120 | 400 |
| | 2 | −15 | 120 | 400 |
| | 3 | 0 | 240 | 400 |
| | 4 | +15 | 180 | 400 |
| | 5 | +25 | 180 | 400 |

| Secondary drying | Secondary set point: +40° C. | | |
|---|---|---|---|
| Post heat setting | Temperature: | Time: | Vacuum: |
| | +25° C. | 600 min | 400 mTorr |

### b) Tablet Compression Step:

All the ingredients were passed thru 30 #ASTM sieve (590 μm).
The freeze dried polymeric powder from above was mixed geometrically with the API and other tableting excipients (except magnesium stearate) as mentioned in Table 1.
Magnesium stearate was added at the end after passing it thru 80 #ASTM sieve (180 μm) to the blend from above step and mixed uniformly.
The blend was compressed into tablets using D tooling fitted on a rotary compression machine.

Process Parameters for Tablet Compression:

| Experiment number | C-13 |
|---|---|
| Punch size (mm) | 12.5 |
| Weight (mg) | 650-655 |
| Hardness (N) | 70-90 |

### 2.4 Process Details for Experiments C-10, C-11 & C-14

### a) Spray Drying and Physical Mixing Step:

EUDRAGIT® FL 30 D was diluted with water to the solids content of 15% (w/w).
The dispersion was then passed thru 60 ASTM sieve (250 μm) and then it was used for spray drying using lab scale Buchi spray drier.

Process Parameters for Spray Drying:

| Experiment number | C-10, C-11 & C-14 |
|---|---|
| Inlet Temperature (° C.) | 50-55 |
| Aspirator (%) | 90-95 |

The spray dried polymeric powder from above step was mixed uniformly with celluloses and passed thru 30 #ASTM sieve (590 microns)

### b) Tablet Compression Step:

All the ingredients were passed thru 30 #ASTM sieve (590 μm).
The spray dried and physically mixed polymeric powder from above was mixed geometrically with the API and other tableting excipients (except magnesium stearate) as mentioned in Table 1.
Magnesium stearate was added at the end after passing it thru 80 #ASTM sieve (180 μm) to the blend from above step and mixed uniformly.
The blend was compressed into tablets using D tooling fitted on a rotary compression machine.

Process Parameters for Tablet Compression:

| Experiment number | C-10, C-11 & C-14 |
|---|---|
| Punch size (mm) | 12.5 |
| Weight (mg) | 650-655 |
| Hardness (N) | 70-90 |

### 2.5 Analysis of the Compressed Tablets

a) Dissolution: All the samples were studied for dissolution profile (24 hrs) using recommended dissolution equipment and media.
b) Curing study: All the compressed tablet samples were studied for curing effect by exposing it to 40° C. for 24 hrs. Dissolution profiles before and after curing were compared using f2 value.
c) Stability: The uncured samples were packed in HDPE containers and charged in a stability chamber maintained at 40° C./75% RH. The samples were analyzed at least after 1 month of storage and studied for any change in dissolution profile using f2 test.

### 2.6 Results of all the Experiments (C-8 to C-14)

| | Experiment numbers | | | | | | |
|---|---|---|---|---|---|---|---|
| Parameters | C-8 | C-9 | C-10 | C-11 | C-12 | C-13 | C-14 |
| No Curing effect on | 98.8 | 89.4 | 61.6 | 81.5 | 96 | 78 | 58.8 |
| dissolution profile | | | | | | | |
| Acceptance criteria: f2 > 50 | | | | | | | |
| Effect on dissolution | Not done | Not done | 34.1 | 32.5 | Not done | 46.3 | 49.4 |
| profile after charging on | | | | | | | |
| accelerated Stability | | | | | | | |
| condition | | | | | | | |
| Acceptance criteria: f2 > 50 | | | | | | | |
| Dissolution profile | 28.33 | 27.70 | 99 | 105 | 29.03 | 94.88 | 109 |
| Acceptance criteria: | | | | | | | |
| NLT 60% in 24 hrs, pH | | | | | | | |
| 6.8 | | | | | | | |
| Batch passing all the | No | No | No | No | No | No | No |
| acceptance criterion | | | | | | | |

### 2.7 Important Findings from Above Experiments (C-8 to C-14)

a) EUDRAGIT® FL 30 D-55 when combined with various non-cellulosic polymers, none of the combination was able to fulfill all the acceptance criteria.
b) All the non-cellulosic combinations with EUDRAGIT® FL 30 D-55 are comparative examples.

1: A powder composition, comprising:
50 to 95% by weight of a copolymer mixture A of
a copolymer 1, comprising 5 to 60% by weight of polymerized units of methacrylic acid and 95 to 40% by weight of a C₁- to C₄-alkylester of (meth)acrylic acid, and
a copolymer 2, comprising more than 95 and up to 100% by weight of polymerized units of a C₁- to C₄-alkylester of (meth)acrylic acid, and
50 to 5% by weight of a water-soluble cellulose B.

2: The powder composition according to claim 1, wherein the copolymer mixture A comprises the copolymer 1 and the copolymer 2 as a mixture of separate copolymers 1 and 2 or as a mixture in a form of a core-shell copolymer.

3: The powder composition according to claim 1, wherein the copolymer 1 comprises polymerized units of 40 to 60% by weight of the methacrylic acid and 60 to 40% by weight of ethyl acrylate or methyl methacrylate.

4: The powder composition according to claim 1, wherein the copolymer 2 comprises polymerized units of 60 to 80% by weight of ethyl acrylate and 40 to 20% by weight of methyl methacrylate.

5: The powder composition according to claim 1, wherein the copolymer mixture A is present in a form of a core-shell polymer with 50 to 90% by weight of a core of the copolymer 2, and 50 to 10% by weight of a shell of the copolymer 1.

6: The powder composition according to claim 1, wherein the water-soluble cellulose B is methyl cellulose, hydroxyethyl cellulose, hydroxyethyl methyl cellulose, hydroxypropyl cellulose, and/or hydroxypropyl methylcellulose.

7: The powder composition according to claim 1, wherein a viscosity of the water-soluble cellulose B is from 1 to 5,000 mPa·s, when measured as 1% aqueous solution or colloidal dispersion (weight/weight) at 25° C.

8: A process for preparing a compressed dosage form comprising the powder composition according to claim 1, the method comprising:
i) providing an aqueous dispersion of the copolymer mixture A and the water-soluble cellulose B,
ii) drying the aqueous dispersion, to gain the powder composition,
iii) mixing the powder composition with one or more biologically active ingredient(s) and one or more pharmaceutical or nutraceutical excipient(s), to gain a mixture for compression, and
iv) compressing the mixture for compression in a form, to obtain the compressed dosage form.

9: The process according to claim 8, wherein in ii), the drying is spray drying performed at an inlet temperature of 30 to 60° C.

10: The process according to claim 8, wherein in ii), the drying is freeze drying performed with a drying circle step for 4 to 16 hours at 350 to 450 mTorr, starting from −40 to −25° C. and increasing stepwise or continuously to a final temperature of 15 to 30° C.

11: A compressed dosage form, comprising one or more pharmaceutically or nutraceutically active ingredient(s), the powder composition according to claim 1, and one or more pharmaceutical or nutraceutical excipient(s).

12: The compressed dosage form according to claim 11, comprising 1 to 50% by weight of the one or more pharmaceutically or nutraceutically active ingredient(s), 10 to 70% by weight of the powder composition according to claim 1, and 10 to 89% by weight of the one or more pharmaceutical or nutraceutical excipient(s).

13: The compressed dosage form according to claim 11, wherein each of a plurality of the compressed dosage forms of a same size, form and composition, with or without curing at 40° C. for 24 hours, has a similarity factor f₂ of 50 or more of an active ingredient release profile from a drug dissolution test at pH 6.8.

14: The compressed dosage form according to claim 11, wherein each of a plurality of the compressed dosage forms of a same size, form and composition, with or without storing in HDPE containers at 40° C. and 75% relative humidity for one month, has a similarity factor f₂ of 50 or more of an active ingredient release profile from a drug dissolution test at pH 6.8.

15: The compressed dosage form according to claim 11, showing an active ingredient release of 60% or more within 24 hours in a dissolution test at pH 6.8.

16: The process according to claim 8, wherein the compressed dosage form is a tablet.

17: The process according to claim 8, wherein in ii), the drying is spray drying or freeze drying.

18: The process according to claim 9, wherein the spray drying is performed at an inlet temperature of 35 to 55° C.

19: The compressed dosage form according to claim 11, wherein the compressed dosage form is a tablet.
